(19) United States
(12) Patent Application Publication
(10) Pub. No.: US 2023/0043049 A1
(43) Pub. Date: Feb. 9, 2023

(54) MOBILE CONFIGURABLE LIGHT THERAPY DEVICE

(71) Applicant: Photonic Healing, LLC DBA Visum Light, Kuna, ID (US)

(72) Inventor: Sharon Katzke, Kuna, ID (US)

(21) Appl. No.: 17/397,487

(22) Filed: Aug. 9, 2021

(51) Int. Cl. A61N 5/06

(52) U.S. Cl. CPC A61N 5/0616; A61N 5/0618; A61N 2005/0626; A61N 2005/0632; A61N 2005/0652; A61N 2005/0659; A61N 2005/0663

(57) ABSTRACT

A handheld light therapy device may include a plurality of LEDs selected from a group of colors including red, green, blue, and near infrared, a first and second end, and a light color controller. The first end includes a handle for holding the apparatus and the second end includes the plurality of LEDs. The light color controller receives a light color signal from a user and outputs a polychromatic light corresponding to the light color input signal from at least two of the plurality of LEDs.

### FIELD

This invention relates to medical devices and more particularly relates to light therapy.

### BACKGROUND

Light therapy devices have various applications in the medical field, including but not limited to the treatment of acne treatment, wrinkled skin, sun-damaged skin, malignant skin cancer growths, psoriasis, and depression.

### SUMMARY

An apparatus for a mobile configurable light therapy device is disclosed.

A handheld light therapy, in one embodiment, includes a plurality of light emitting diodes (“LEDs”) selected from a group of colors including red, green, blue, and near infrared, a first and second end, and a light color controller. In one embodiment, the first end includes a handle for holding the apparatus and the second end includes the plurality of LEDs. In one embodiment, the light color controller receives a light color signal from a user and outputs a polychromatic light corresponding to the light color input signal from at least two of the plurality of LEDs.

In some embodiments, the plurality of LEDs are substantially aligned to emit light in the same direction.

In some embodiments, the handheld light therapy apparatus also includes an irradiance controller that receives a selected irradiance and outputs light through at least one LED of the plurality of LEDs at the selected irradiance.

In some embodiments, the handheld light therapy apparatus also includes a timer controller that receives a selected time and outputs light through at least one LED of the plurality of LEDs until the selected time expires.

In some embodiments, the light is output for a pre-determined amount of time.

In some embodiments, the handheld light therapy apparatus also includes an energy density controller that receives a selected energy density and outputs light through at least one LED of the plurality of LEDs at an irradiance corresponding to the selected energy density for the pre-determined amount of time.

In other embodiments, the energy density controller receives a selected energy density and outputs light through at least one LED of the plurality of LEDs based on a selected time or a selected irradiance until the selected energy density is reached.

In some embodiments, the selected energy density is within the range of 1 j/cm² to 80 j/cm².

In some embodiments, the handheld light therapy apparatus also includes a frequency controller that comprises a plurality of pre-programmed frequencies, wherein the frequency controller receives a frequency signal selection corresponding to a pre-programmed frequency of the plurality of pre-programmed frequencies and pulses light from at least one LED of the plurality of LEDs at the pre-programmed frequency corresponding to the frequency signal.

In some embodiments, the pre-programmed frequency comprises a Nogier frequency.

In some embodiments, the light color input signal is selected from a group including red, green, blue, near infrared, magenta, turquoise, and white.

A light therapy apparatus, in one embodiment, includes a plurality of LEDs selected from a group of colors comprising red, green, blue, and near infrared, and an energy density controller that receives a selected energy density and outputs light through at least one light emitting diode of the plurality of light emitting diodes based on a selected time or a selected irradiance until the selected energy density is reached.

In some embodiments, the LEDs are substantially aligned to emit light in the same direction.

In some embodiments, the light therapy apparatus also includes an irradiance controller that receives a selected irradiance and outputs light through at least one LED of the plurality of LEDs at the selected irradiance.

In some embodiments, the light therapy apparatus also includes a timer controller that receives a selected time and outputs light through at least one LED of the plurality of LEDs until the selected time expires.

In some embodiments, the selected energy density is within the range of 1 j/cm² to 80 j/cm².

In some embodiments, the light therapy apparatus also includes a frequency controller that comprises a plurality of pre-programmed frequencies, wherein the frequency controller receives a frequency signal selection corresponding to a pre-programmed frequency of the plurality of pre-programmed frequencies and pulses light from at least one LED of the plurality of LEDs at the pre-programmed frequency corresponding to the frequency signal.

In some embodiments, the pre-programmed frequency comprises a Nogier frequency.

In some embodiments, the light therapy apparatus also includes a light color controller that does one of receive a light color input signal from a user corresponding to one of red, green, blue, and near infrared, and outputs a monochromatic light from the first light emitting diode and receive a light color input signal from the user and outputs a polychromatic light corresponding to the light color input signal from at least two LEDs of the plurality of LEDs, or a combination thereof. In further embodiments, the light color input signal is selected from a group comprising red, green, blue, near infrared, magenta, yellow, turquoise, and white.

An apparatus, in one embodiment, includes a plurality of LEDs, a first end and a second end, a light color controller, an irradiance controller, a timer controller, an energy density controller, and a frequency controller.

In some embodiments, the plurality of LEDs are selected from a group of colors comprising red, green, blue, and near infrared.

In some embodiments, the first end comprises a handle for holding the apparatus and the second end includes the plurality of LEDs.

In some embodiments, the light controller that does one of receive a light color input signal from a user corresponding to one of red, green, blue, and near infrared, and outputs a monochromatic light from the first light emitting diode and receive a light color input signal from the user and outputs a polychromatic light corresponding to the light color input signal from at least two LEDs of the plurality of LEDs, or a combination thereof. In further embodiments, the light color input signal is selected from a group comprising red, green, blue, near infrared, magenta, yellow, turquoise, and white.

In some embodiments, the irradiance controller receives a selected irradiance and outputs light through at least one LED of the plurality of LEDs at the selected irradiance.

In some embodiments, the timer controller receives a selected time and outputs light through at least one LED of the plurality of LEDs until the selected time expires.

In some embodiments, the energy density controller receives a selected energy density and outputs light through at least one light emitting diode of the plurality of light emitting diodes based on a pre-determined time or a pre-determined irradiance until the selected energy density is reached. In certain embodiments, the selected energy density is within the range of 1 j/cm² to 80 j/cm².

In some embodiments, the frequency controller that comprises a plurality of pre-programmed frequencies where the frequency controller receives a frequency signal selection corresponding to a pre-programmed frequency of the plurality of pre-programmed frequencies and pulse light from at least one light emitting diode of the plurality of light emitting diodes at the pre-programmed frequency corresponding to the frequency signal. In certain embodiments, the pre-programmed frequency includes a Nogier frequency.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1A is a perspective view illustrating one embodiment of a handheld mobile configurable light therapy device;

FIG. 1B is a perspective view further illustrating one embodiment of a mobile configurable light therapy handheld apparatus.

FIG. 2 is a perspective view illustrating one embodiment of a mobile configurable light therapy pad;

FIG. 3 is a block diagram illustrating one embodiment of a mobile configurable light therapy device;

FIG. 4 is a flowchart illustrating a certain method of using a mobile configurable light therapy device; and

FIG. 5 is a flowchart illustrating a certain method of using a mobile configurable light therapy device.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Any computer program product described herein may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (“ISA”) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (“LAN”) or a wide area network (“WAN”), or the connection may be made to an external computer (for example, through the Internet using an Internet Service Provider). In some embodiments, electronic circuitry including, for example, programmable logic circuitry, field-programmable gate arrays (“FPGA”), or programmable logic arrays (“PLA”) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program instructions may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

The schematic flowchart diagrams and/or schematic block diagrams in the Figures illustrate the architecture, functionality, and operation of possible implementations of apparatuses, systems, methods and computer program products according to various embodiments of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

As used herein, a list with a conjunction of “and/or” includes any single item in the list or a combination of items in the list. For example, a list of A, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single item in the list. For example, “one of A, B and C” includes only A, only B or only C and excludes combinations of A, B and C. As used herein, “a member selected from the group consisting of A, B, and C,” includes one and only one of A, B, or C, and excludes combinations of A, B, and C.” As used herein, “a member selected from the group consisting of A, B, and C and combinations thereof” includes only A, only B, only C, a combination of A and B, a combination of B and C, a combination of A and C or a combination of A, B and C.

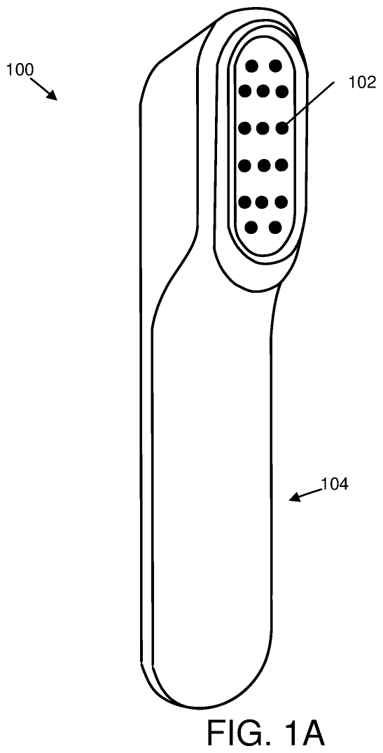

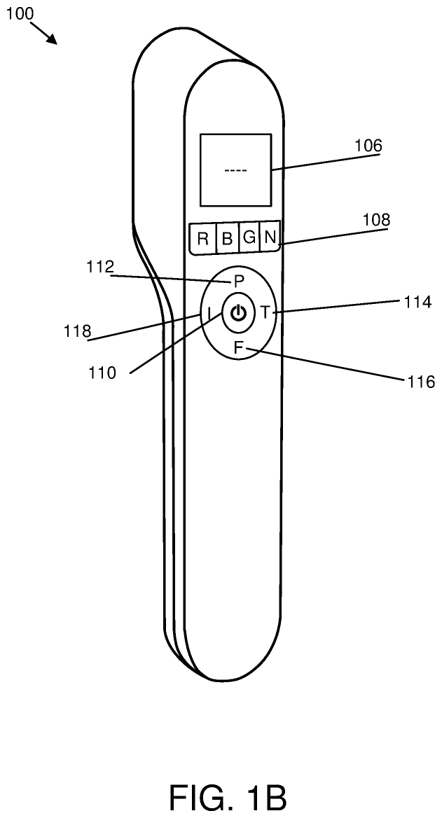

FIG. 1A is a perspective view illustrating one embodiment of a mobile configurable light therapy handheld apparatus 100. As used herein, a mobile configurable light therapy handheld apparatus 100 may refer to a device of the present disclosure that is used for light therapy medical purposes. The mobile configurable light therapy handheld apparatus 100, in one embodiment, includes a plurality of light emitting diodes (“LEDs”) 102 and a handle 104.

In certain embodiments, the LEDs 102 are selected from a group of colors comprising red, green, blue, and near infrared. In some embodiments, the red LEDs emit light at a wavelength of 660 nm, the green LEDs emit light at a wavelength of 535 nm, the blue LEDs emit light at a wavelength of 410 nm, and the near-infrared LEDs emit light at a wavelength of 810 nm. In some embodiments, there are 16 total LEDs, four of each of the colors red, green, blue, and near infrared. In one embodiment, the LEDs 102 are substantially aligned to emit light in the same direction and are protected by a tempered glass lens. In some embodiments, the handheld apparatus 100 further includes at least one rechargeable battery and a battery charging port.

In some embodiments, the LEDs 102 may emit monochromatic light corresponding to the group of colors red, green, blue, and near infrared. In certain embodiments, the LEDs may emit polychromatic light (e.g., magenta, yellow, turquoise, white, and so forth) corresponding to a plurality of combined colors selected from a group including red, green, blue, and near infrared.

In some embodiments, the user selects a monochromatic or polychromatic color dependent on what type of healing property the user desires. The color red is designed to energize all organs and senses, increase circulation and blood flow, and release stiffness and constrictions. The color blue is designed to soothe, cool, be anti-bacterial and anti-fungal, and reduce acute inflammation. The color green is designed to balance the mind and body, relieve mental stress, and relieve heart related concerns. The color magenta is designed to balance emotions, level blood pressure, and stimulate kidneys, adrenals, and the heart. The color yellow is designed to aid with digestive issues, support the stomach, liver, and intestines, and stimulate the thymus gland. The color turquoise is designed to tranquilize the mind and spirit, relieve cranial pressure, and increase intuition and sensitivity. The color white is designed to penetrate the skin the deepest, speed up skin metabolism, and reduce fine lines and wrinkles.

In some embodiments, the handle 104 is at an end opposite of the LEDs 102 so that a user may hold the handheld apparatus 100 by the handle 104 at the same time as another part of the user's body is exposed to the emitted light of the LEDs 102. In other embodiments, the handle 104 may include an adhesive surface area or strap to attach to a user so that the handheld apparatus 100 does not need to be held while emitting light onto an exposed surface.

FIG. 1B is a perspective view further illustrating one embodiment of a mobile configurable light therapy handheld apparatus 100. The handheld apparatus 100, in one embodiment, includes an LED screen 106 and a plurality of buttons used to control the apparatus.

In some embodiments, the LED screen 106 shows the current setting of the device including a selected color, a light emission timer, an irradiance amount, and total joules administered for the light therapy session. In other embodiments, the LED screen 106 additionally shows the battery capacity of the handheld apparatus 100.

In some embodiments, color buttons 108 may be used to initiate the emission of red, blue, green, or near infrared light. In those embodiments, monochromatic light may be emitted by selecting only one of the buttons of the color buttons 108 while polychromatic light may be emitted by selecting a plurality of the color buttons 108.

In certain embodiments, a pre-programmed frequency pattern button 112 and a Nogier frequency button 116 may be used to change the pulsing frequency of the emitted light. In some embodiments, a timer button 114 may be used to change the length of the light emission timer. In certain embodiments, an irradiance button 118 may be used to change the irradiance level of the emitted light of the handheld apparatus 100. As used herein, irradiance is used to describe flux of radiant energy per unit area, e.g., an irradiance of 99.9 mW/cm².

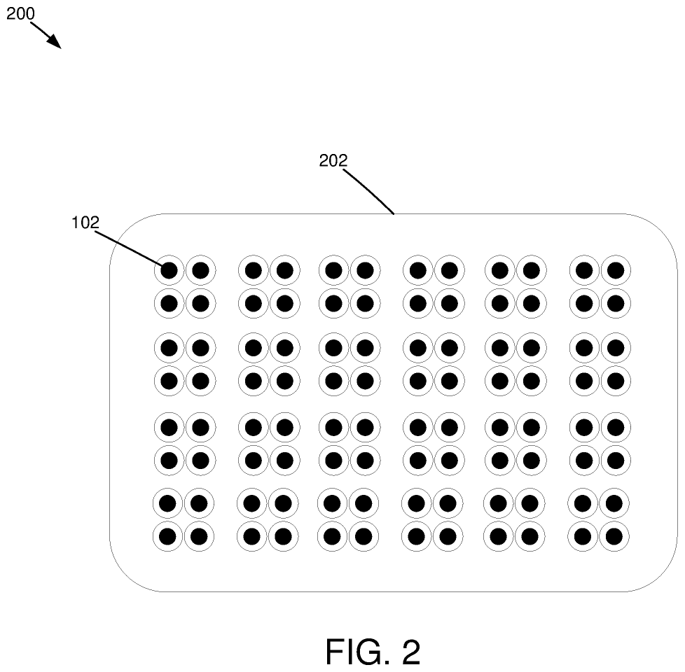

FIG. 2 is a perspective view illustrating one embodiment of a mobile configurable light therapy pad apparatus 200. As used herein, a mobile configurable light therapy pad device 200 may refer to a device of the present disclosure that is used for light therapy medical purposes. The mobile configurable light therapy pad apparatus 200, in one embodiment, includes a plurality of LEDs 102 and a pad 202.

In certain embodiments, the LEDs 102 are selected from a group of colors comprising red, green, blue, and near infrared. In one embodiment, the LEDs 102 are substantially aligned to emit light in the same direction.

In some embodiments, the LEDs 102 may emit monochromatic light corresponding to the group of colors red, green, blue, and near infrared. In certain embodiments, the LEDs may emit polychromatic light (e.g., magenta, yellow, turquoise, white, and so forth) corresponding to a plurality of combined colors selected from a group including red, green, blue, and near infrared.

In some embodiments, the pad 202 is made of a flexible material that can be molded to best suit the light therapy treatment area. In other embodiments, the pad 202 may include an adhesive surface area or strap to attach to a user to that the pad may be secured against the light therapy treatment area without additional support.

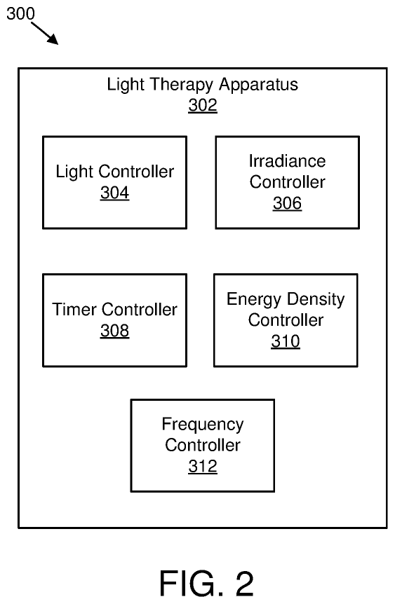

FIG. 3 is a block diagram illustrating one embodiment of a mobile configurable light therapy device 300. The light therapy device 300 includes a light therapy apparatus 302, wherein the light therapy apparatus 302 comprises one or more of a light controller 304, an irradiance controller 306, a timer controller 308, an energy density controller 310, and a frequency controller 312.

In some embodiment, the light therapy apparatus 302 may comprise the mobile configurable light therapy handheld apparatus 100. In certain embodiments, the light therapy apparatus 302 may comprise the mobile configurable light therapy pad apparatus 200. In other embodiments, the light therapy apparatus 302 may comprise of a plurality of LEDs 102 selected from a group of colors comprising red, green, blue, and near infrared, where the LEDs 102 are housed within a device typical of light therapy devices, such as a light box, full-body lamp, or light bed.

In some embodiments, the light controller 304 receives a light color input signal from a user corresponding to one of red, green, blue, and near infrared, and outputs a monochromatic light from at least one of the LEDs 102. In certain embodiments, the light controller 304 receives a light color input signal from a user and outputs a polychromatic light (e.g., magenta, yellow, turquoise, white, and so forth) corresponding to the light color input signal from at least two LEDs 102. In one embodiment, the light color input signal is selected from a group comprising red, green blue, near infrared, magenta, yellow, turquoise, and white. In other embodiments, the light color input signal is selected from any color within the visible light spectrum as well as some non-visible selections.

In some embodiments, the irradiance controller 306 receives a selected irradiance and outputs light through at least one LED 102 at the selected irradiance. In certain embodiments, the irradiance controller 306 may include a pre-determined selected irradiance that is used in the absence of a user-chosen selected irradiance. In some embodiments, the selected irradiance—sometimes known as power intensity—is selected from the group of 33.3 mW/cm², 59.2 mW/cm², 66.6 mW/cm², 67.2 mW/cm², 99.9 mW/cm², and 133.2 mW/cm². In other embodiments, the selected irradiance is less than or equal to 133.2 mW/cm². In certain embodiments, selected irradiances 33.3 mW/cm² and 66.6 mW/cm² correspond to red, blue, green, and near infrared light of the light controller 304. In some embodiments, selected irradiances 99.9 mW/cm² and 133.2 mW/cm² correspond to red, blue, and near infrared light of the light controller 304. In some embodiments, a selected irradiance of 33.3 mW/cm² corresponds to green light of the light controller 304. In certain embodiments, the selected irradiance corresponding to the color green supersedes other selected irradiances when green light, either as a monochromatic emission or part of a polychromatic emission, is emitted by the light controller.

In some embodiments, the timer controller 308 receives a selected time and outputs light through at least one LED 102 until the selected time expires. In certain embodiments, the timer controller 308 may include a pre-determined selected time that is used in the absence of a user-chosen selected time. In some embodiments, the user may select a time from a group of times ranging from 30 seconds to ten minutes with 30-second intervals in between.

In some embodiments, the energy density controller 310 receives and applies a selected energy density through use of the time and irradiance inputs and outputs light through at least one LED 102 until the selected energy density is reached. For example, the energy density controller may use a selected time of ten minutes and a selected irradiance of 66.6 mW/cm² to select an energy density of 40 J/cm². In certain embodiments, the energy density controller 310 is used in tandem with a pre-determined irradiance. In these embodiments, the pre-determined selected irradiance is used with a selected time from the timer controller 308 to achieve the desired energy density. In some embodiments, the energy density controller 310 is used in tandem with a pre-determined selected time. In these embodiments, the pre-determined selected time is used with a selected irradiance from the irradiance controller 306 to achieve the desired energy. In certain embodiments, a selected energy density may be used with one or more of a user-chosen selected irradiance or a user-chosen selected time. In some embodiments, the selected energy density is within the range of 1 j/cm² to 80 j/cm². In other embodiments, the selected energy density may be less than 1 j/cm² or more than 80 j/cm².

In some embodiments, the frequency controller 312 includes a plurality of pre-programmed frequencies, wherein the frequency controller receives a frequency signal selection corresponding to a pre-programmed frequency of the plurality of pre-programmed frequencies and pulses light from at least one LED 102 at the pre-programmed frequency corresponding to the frequency signal. In certain embodiments, at least one pre-programmed frequency is a Nogier frequency. In some embodiments, the frequency controller 312 may include a pre-determined selected frequency that is used in the absence of a user-chosen selected frequency. In certain embodiments, the pre-determined frequencies may be within the range of 10 Hz to 4.672 KHz. In other embodiments, the pre-determined frequencies may be less than 10 Hz or more than 4.672 KHz. In some embodiments, a user may select a frequency from a group a frequencies including 10 Hz, 40 Hz, 292 Hz, 584 Hz, 1168 Hz, 2336 Hz, 4672 Hz, 73 Hz, and 146 Hz. In some embodiments, the frequency controller 312 also includes a continuous direct dosing where the light from the LEDs 102 does not pulse. In certain embodiments, certain frequencies may be selected to be directed at pain, anxiety, skin, energy, and the brain. In some embodiments, the effects of Nogier frequencies of the frequency controller 312 are directed towards action on the tissues including wounds and epidermal conditions, gastrointestinal and metabolic problems, locomotor problems, disorders of laterality, pain in nerve conduction, brain and bone reconstruction and healing, and action on the cerebral cortex including cortical and mental disorders.

In some embodiments, there may be certain combinations of a frequency of the frequency controller 312, a light color of the light controller 304, and a location where the light is applied. For example, a combination of a 10 Hz frequency and a red, blue, and/or near infrared color may be applied directly to an area of concern and the belly button. As another example, a combination of a 40 Hz frequency and a red, blue, and/or near infrared color may be applied directly to an area of concern and the belly button.

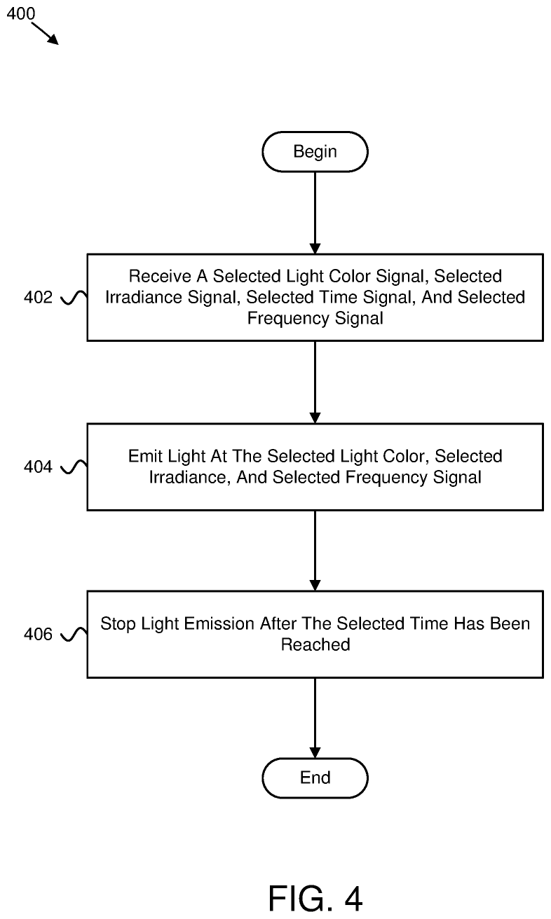

FIG. 4 depicts a schematic flow-chart diagram illustrating one embodiment of a method 400 for using a light therapy apparatus. In one embodiment, the method 400 begins and receives 402 a selected light color signal, a selected irradiance signal, selected time signal, and selected frequency signal. In certain embodiments, the method 400 emits 404 light at the selected light color, at the selected irradiance, and at the selected frequency signal. In certain embodiments, the method 400 stops 406 light emission after the selected time has been reached, and the method ends.

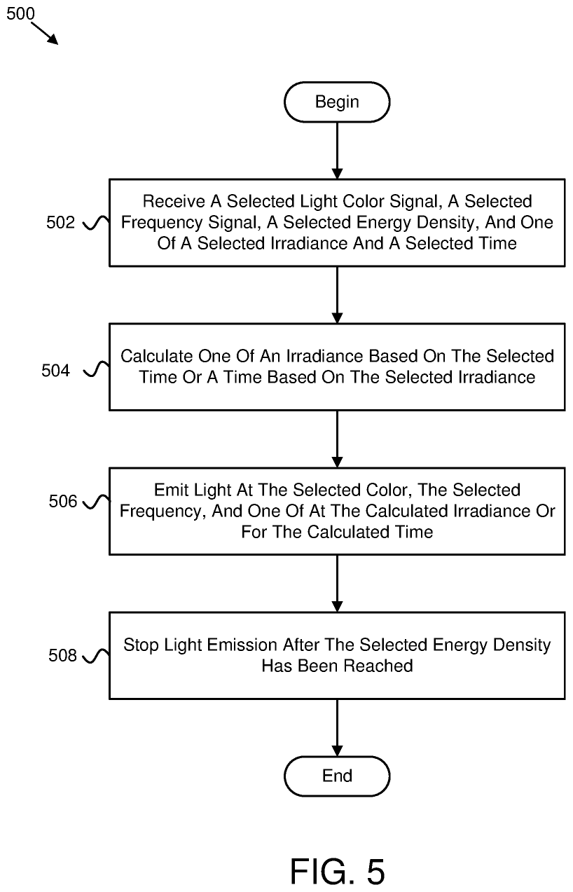

FIG. 5 depicts a schematic flow-chart diagram illustrating one embodiment of a method 500 for using a light therapy apparatus including an energy density controller. In one embodiment, the method 500 begins and receives 502 a selected light color signal, a selected frequency signal, a selected energy density, and one of a selected irradiance and a selected time. In certain embodiments, the method 500 calculates 504 one of an irradiance based on the selected time or a time based on the selected irradiance. In certain embodiments, the method 500 emits 506 light at the selected color, the selected frequency, and one of at the calculated irradiance or for the calculated time. In certain embodiments, the method 500 stops 508 light emission after the selected energy density has been reached.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A handheld apparatus comprising:
a plurality of light emitting diodes selected from a group of colors comprising red, green, blue, and near infrared;
a first end and a second end, wherein the first end comprises a handle for holding the handheld apparatus and the second end comprises the plurality of light emitting diodes; and
a light color controller that receives a light color input signal from the user and outputs a polychromatic light corresponding to the light color input signal from at least two light emitting diodes of the plurality of light emitting diodes.

2. The apparatus of claim 1, wherein the plurality of light emitting diodes are substantially aligned to emit light in the same direction.

3. The apparatus of claim 1, further comprising an irradiance controller that receives a selected irradiance and outputs light through at least one light emitting diode of the plurality of light emitting diodes at the selected irradiance.

4. The apparatus of claim 3, further comprising a timer controller that receives a selected time and outputs light through at least one light emitting diode of the plurality of light emitting diodes until the selected time expires.

5. The apparatus of claim 3, wherein the light is output for a pre-determined amount of time.

6. The apparatus of claim 5, further comprising an energy density controller that receives a selected energy density and outputs light through at least one light emitting diode of the plurality of light emitting diodes at an irradiance corresponding to the selected energy density for the pre-determined amount of time.

7. The apparatus of claim 1, further comprising an energy density controller that receives a selected energy density and outputs light through at least one light emitting diode of the plurality of light emitting diodes based on a selected time or a selected irradiance until the selected energy density is reached.

8. The apparatus of claim 6, wherein the selected energy density is within the range of 1 j/cm² to 80 j/cm².

9. The apparatus of claim 1, further comprising a frequency controller that comprises a plurality of pre-programmed frequencies, wherein the frequency controller receives a frequency signal selection corresponding to a pre-programmed frequency of the plurality of pre-programmed frequencies and pulses light from at least one light emitting diode of the plurality of light emitting diodes at the pre-programmed frequency corresponding to the frequency signal.

10. The apparatus of claim 9, wherein the pre-programmed frequency comprises a Nogier frequency.

11. The apparatus of claim 1, wherein the light color input signal is selected from a group comprising red, green, blue, near infrared, magenta, yellow, turquoise, and white.

12. An apparatus comprising:
a plurality of light emitting diodes selected from a group of colors comprising red, green, blue, and near infrared; and
an energy density controller that receives a selected energy density and outputs light through at least one light emitting diode of the plurality of light emitting diodes based on a selected time or a selected irradiance until the selected energy density is reached.

13. The apparatus of claim 12, wherein the light emitting diodes are substantially aligned to emit light in the same direction.

14. The apparatus of claim 12, further comprising an irradiance controller that receives a selected irradiance and outputs light through at least one light emitting diode of the plurality of light emitting diodes at the selected irradiance.

15. The apparatus of claim 14, further comprising a timer controller that receives a selected time and outputs light through at least one light emitting diode of the plurality of light emitting diodes until the selected time expires.

16. The apparatus of claim 12, wherein the selected energy density is within the range of 1 j/cm² to 80 j/cm².

17. The apparatus of claim 12, further comprising a frequency controller that comprises a plurality of pre-programmed frequencies, wherein the frequency controller receives a frequency signal selection corresponding to a pre-programmed frequency of the plurality of pre-programmed frequencies and pulses light from at least one light emitting diode of the plurality of light emitting diodes at the pre-programmed frequency corresponding to the frequency signal.

18. The apparatus of claim 17, wherein the pre-programmed frequency comprises a Nogier frequency.

19. The apparatus of claim 12, further comprising a light color controller that:
receives a light color input signal from a user corresponding to one of red, green, blue, and near infrared, and outputs a monochromatic light from the first light emitting diode;
receives a light color input signal from the user and outputs a polychromatic light corresponding to the light color input signal from at least two light emitting diodes of the plurality of light emitting diodes;
or a combination thereof;
wherein the light color input signal is selected from a group comprising red, green, blue, near infrared, magenta, yellow, turquoise, and white.

20. An apparatus comprising:
a plurality of light emitting diodes selected from a group of colors comprising red, green, blue, and near infrared;
a first end and a second end, wherein the first end comprises a handle for holding the handheld apparatus and the second end comprises the plurality of light emitting diodes;
a light color controller that:
receives a light color input signal from a user corresponding to a first light emitting diode of the plurality of light emitting diodes and outputs a monochromatic light from the first light emitting diode;
receives a light color input signal from the user and outputs a polychromatic light corresponding to the light color input signal from at least two light emitting diodes of the plurality of light emitting diodes;
or a combination thereof;
an irradiance controller that receives a selected irradiance and outputs light through at least one light emitting diode of the plurality of light emitting diodes at the selected irradiance;
a timer controller that receives a selected time and outputs light through at least one light emitting diode of the plurality of light emitting diodes until the selected time expires;
an energy density controller that receives a selected energy density and outputs light through at least one light emitting diode of the plurality of light emitting diodes based on a pre-determined time or a pre-determined irradiance until the selected energy density is reached;
wherein the selected energy density is within the range of 1 j/cm² to 80 j/cm²; and
a frequency controller that comprises a plurality of pre-programmed frequencies, wherein the frequency controller receives a frequency signal selection corresponding to a pre-programmed frequency of the plurality of pre-programmed frequencies and pulses light from at least one light emitting diode of the plurality of light emitting diodes at the pre-programmed frequency corresponding to the frequency signal;
wherein the pre-programmed frequency comprises a Nogier frequency.
